(19) United States
(12) Patent Application Publication
(10) Pub. No.: US 2026/0053466 A1
(43) Pub. Date: Feb. 26, 2026

(54) SYSTEM FOR DETERMINING INFORMATION RELATED TO CARDIAC DEFORMATION OF AN ANATOMICAL FEATURE OF A HEART USING DEFORMATION IMAGING

(71) Applicant: GE Precision Healthcare LLC, Waukesha, WI (US)

(72) Inventor: Hani Nozari MIRAR, Oslo (NO)

(21) Appl. No.: 18/809,947

(22) Filed: Aug. 20, 2024

(51) Int. Cl. A61B 8/08; A61B 8/00; G06T 7/00; G06T 7/11

(52) U.S. Cl. CPC A61B 8/0883; A61B 8/463; A61B 8/469; A61B 8/483; A61B 8/485; G06T 7/0012; G06T 7/11; G06T 2207/10136; G06T 2207/20021; G06T 2207/20096; G06T 2207/30048

(57) ABSTRACT

Various systems and methods are provided for determining information related to cardiac deformation of an anatomical feature of a heart of a subject using deformation imaging and a plurality of overlapping segments that extend along a boundary of the anatomical feature of the heart of the subject. Imaging data of the anatomical feature of the heart of the subject may be received. A boundary of the anatomical feature of the heart of the subject may be delineated. The anatomical feature may be divided into a plurality of overlapping segments that extend along the boundary of the anatomical feature of the heart of the subject. Information related to cardiac deformation of the anatomical feature of the heart of the subject may be determined using deformation imaging and the plurality of overlapping segments. The information related to cardiac deformation of the anatomical feature of the heart may be displayed.

### TECHNICAL FIELD

The present disclosure relates to a system and method for determining information related to cardiac deformation of one or more anatomical features of a heart of a subject using deformation imaging and a plurality of overlapping segments that extend along a boundary of the anatomical feature of the heart of the subject. Further, the present disclosure relates to a system and method for determining mechanical dyssynchrony through the enhanced determination of mechanical dispersion.

### BACKGROUND

Deformation imaging may refer to an imaging technique to evaluate myocardial deformation. Deformation, or “strain,” may refer to the change in cardiac length of the myocardium from end-diastole to end-systole. Deformation imaging may be used for assessment of myocardial mechanics. For example, deformation imaging may be used to detect mechanical dyssynchrony, cardiomyopathies, heart disease, myocardial dysfunction, or the like.

For deformation imaging, an anatomical feature of the heart may be divided into a set of segments. The segments may be tracked during the cardiac cycle using a deformation imaging technique (e.g., a template matching technique, an image registration technique, an artificial intelligence (AI) technique, or the like). Strain values for the set of segments may be determined based on the tracking of the segments. For example, a strain curve, or “strain trace,” that includes strain values for a segment over the cardiac cycle may be determined. Various relevant strain values (e.g., end-systolic strain, peak systolic strain, peak strain, etc.) may be determined from the strain curve.

Each segment may require a different amount of time to reach a maximum strain value. The standard deviation of the different amounts of time to reach maximum strain values may be referred to as “mechanical dispersion. ” Put another way, mechanical dispersion of the left atrium of the heart may refer to the variability in the timing of atrial contraction. In some cases, mechanical dispersion measurements may enhance the accuracy of determining cardiac mechanical dyssynchrony.

In some cases, a deformation imaging system may use a fixed number of non-overlapping segments of an anatomical feature when determining strain values for the anatomical feature, which can adversely impact the true value of mechanical dispersion. In this way, the deformation imaging system might inaccurately, or erroneously, determining mechanical dispersion of the anatomical feature, and/or might inaccurately, or erroneously, determine cardiac mechanical dyssynchrony.

### SUMMARY

This summary introduces concepts that are described in more detail in the detailed description. It should not be used to identify essential features of the claimed subject matter, nor to limit the scope of the claimed subject matter.

In an aspect, a system may include a memory configured to store instructions; and one or more processors configured to execute the instructions to: receive imaging data of an anatomical feature of a heart of a subject; delineate a boundary of the anatomical feature of the heart of the subject; divide the anatomical feature into a plurality of overlapping segments that extend along the boundary of the anatomical feature of the heart of the subject; determine information related to cardiac deformation of the anatomical feature of the heart of the subject using deformation imaging and the plurality of overlapping segments; and display the information related to cardiac deformation of the anatomical feature of the heart.

In another aspect, a method may include receiving imaging data of an anatomical feature of a heart of a subject; delineating a boundary of the anatomical feature of the heart of the subject; dividing the anatomical feature into a plurality of overlapping segments that extend along the boundary of the anatomical feature of the heart of the subject; determining information related to cardiac deformation of the anatomical feature of the heart of the subject using deformation imaging and the plurality of overlapping segments; and displaying the information related to cardiac deformation of the anatomical feature of the heart.

In yet another aspect, a non-transitory computer-readable medium may store instructions that, when executed by one or more processors, cause the one or more processors to: receive imaging data of an anatomical feature of a heart of a subject; delineate a boundary of the anatomical feature of the heart of the subject; divide the anatomical feature into a plurality of overlapping segments that extend along the boundary of the anatomical feature of the heart of the subject; determine information related to cardiac deformation of the anatomical feature of the heart of the subject using deformation imaging and the plurality of overlapping segments; and display the information related to cardiac deformation of the anatomical feature of the heart.

### BRIEF DESCRIPTION OF DRAWINGS

FIG. 1 is a diagram of an example system for determining information related to cardiac deformation of an anatomical feature of a heart of a subject using deformation imaging and a plurality of overlapping segments that extend along a boundary of the anatomical feature of the heart of the subject.

FIG. 2 is a diagram of an example deformation imaging system for determining information related to cardiac deformation of an anatomical feature of a heart of a subject using deformation imaging and a plurality of overlapping segments that extend along a boundary of the anatomical feature of the heart of the subject.

FIG. 3 is a diagram of an example ultrasound system for acquiring ultrasound data of the heart of the subject.

FIG. 4 is a diagram of an example preoperative imaging system for acquiring preoperative imaging data of the heart of the subject.

FIG. 5 is a flowchart of an example process for determining information related to cardiac deformation of an anatomical feature of a heart of a subject using deformation imaging and a plurality of overlapping segments that extend along a boundary of the anatomical feature of the heart of the subject.

FIG. 6 is a diagram of an example user interface that displays an ultrasound image and an anatomical feature with a delineated boundary.

FIG. 7 is a diagram of an example user interface that displays an ultrasound image, an anatomical feature with a delineated boundary, and a plurality of overlapping segments.

FIG. 8 is a diagram of an example user interface that displays strain traces that display respective strain values of a set of corresponding segments over time, and time plots that display respective amounts of time to reach maximum strain values for the set of corresponding segments.

FIG. 9 is a diagram of an example user interface that displays an ultrasound image and respective tracking quality indicators of a set of segments, and that displays strain traces that display respective strain values of a set of corresponding segments over time.

FIG. 10 is a diagram of an example user interface that displays ultrasound images corresponding to respective apical planes of the heart which delineates an endocardial border of the left atrium.

FIG. 11 is a diagram of an example user interface that displays ultrasound images displaying respective markers of segments associated with amounts of time to reach maximum strain value that are greater than or less than respective time thresholds, and that displays a model of the anatomical feature of the heart that displays the markers.

FIG. 12 is a diagram of an example user interface that displays an ultrasound image displaying respective markers of segments that identify respective times of the corresponding segments to reach peak strain values.

FIG. 13 is a diagram of an example user interface that displays ultrasound images displaying multiple anatomical features of the heart, and that displays a model displaying the multiple anatomical features of the heart.

FIG. 14 is a diagram of an example user interface that displays an ultrasound image with markers that highlights areas of longest contraction or conduction delay of the heart.

### DETAILED DESCRIPTION

As addressed above, a deformation imaging system may delineate a fixed number of non-overlapping segments of an anatomical feature, and determine strain values for the fixed number of non-overlapping segments. Further, the deformation imaging may determine a mechanical dispersion value based on a standard deviation of the strain values. Further still, the deformation imaging system may determine cardiac mechanical dyssynchrony of the heart based on the mechanical dispersion value. However, the mechanical dispersion value may be inaccurate due to the purely geometrical division of cardiac muscle into a fixed number of non-overlapping segments. This method may not accurately represent the underlying segment boundaries.

Some embodiments of the present disclosure are directed to a system configured to receive imaging data of an anatomical feature of a heart of a subject; delineate a boundary of the anatomical feature of the heart of the subject; divide the anatomical feature into a plurality of overlapping segments that extend along the boundary of the anatomical feature of the heart of the subject; determine information related to cardiac deformation of the anatomical feature of the heart of the subject using deformation imaging and the plurality of overlapping segments; and display the information related to cardiac deformation of the anatomical feature of the heart.

In contrast to using the predefined and fixed number of segments, the present disclosure provides the utilization of the plurality of overlapping segments that extend along the boundary of the anatomical feature of the heart of the subject. In this way, the present disclosure improves the precision of the determination of mechanical dispersion, improves the precision of the determination of information related to cardiac deformation, is robust against noisy measurements, offers improved localization of regions with contraction delay, and improves the reliability of the determination of mechanical dispersion. Further, in this way, the present disclosure may benefit patients by facilitating the early diagnosis of cardiac pathologies. Accordingly, the present disclosure provides an improvement to the technical field of cardiac deformation imaging, and provides a technical improvement to cardiac deformation imaging systems by providing more accurate determinations of, among other things, mechanical dispersion and cardiac dyssynchrony.

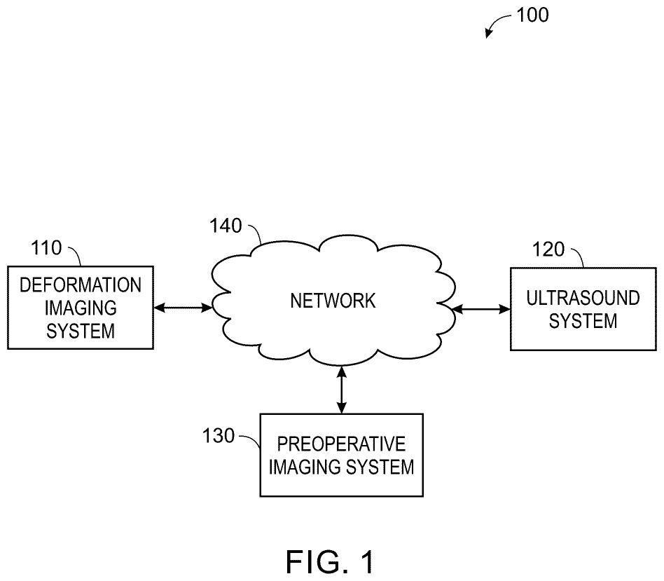

FIG. 1 is a diagram of an example system for determining information related to cardiac deformation of an anatomical feature of a heart of a subject using deformation imaging and a plurality of overlapping segments that extend along a boundary of the anatomical feature of the heart of the subject. As shown in FIG. 1, the system 100 may include a deformation imaging system 110, an ultrasound system 120, a preoperative imaging system 130, and a network 140.

The deformation imaging system 110 may be configured to receive imaging data of an anatomical feature of a heart of a subject, delineate a boundary of the anatomical feature of the heart of the subject, divide the anatomical feature into a plurality of overlapping segments that extend along the boundary of the anatomical feature of the heart of the subject, determine information related to cardiac deformation of the anatomical feature of the heart of the subject using deformation imaging and the plurality of overlapping segments, and display the information related to cardiac deformation of the anatomical feature of the heart. For example, the deformation imaging system 110 may be a computer, a server, a medical device, or the like.

The ultrasound system 120 may be configured to acquire ultrasound data of a region of interest of a heart of a subject. For example, the ultrasound system 120 may be a two-dimensional (2D) ultrasound system, a three-dimensional (3D) ultrasound system, a four-dimensional (4D) ultrasound system, a Doppler ultrasound system, or the like. The subject may be a person, an animal, a phantom, or the like.

The preoperative imaging system 130 may be configured to acquire preoperative imaging data of the heart of the subject. For example, the preoperative imaging system 130 may be a computed tomography (CT) system, a magnetic resonance imaging (MRI) system, an ultrasound system, an X-ray system, a positron emission tomography (PET) device, or the like.

The network 140 may permit communication between the deformation imaging system 110, the ultrasound system 120, and the preoperative imaging system 130. For example, the network 140 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The number and arrangement of the system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

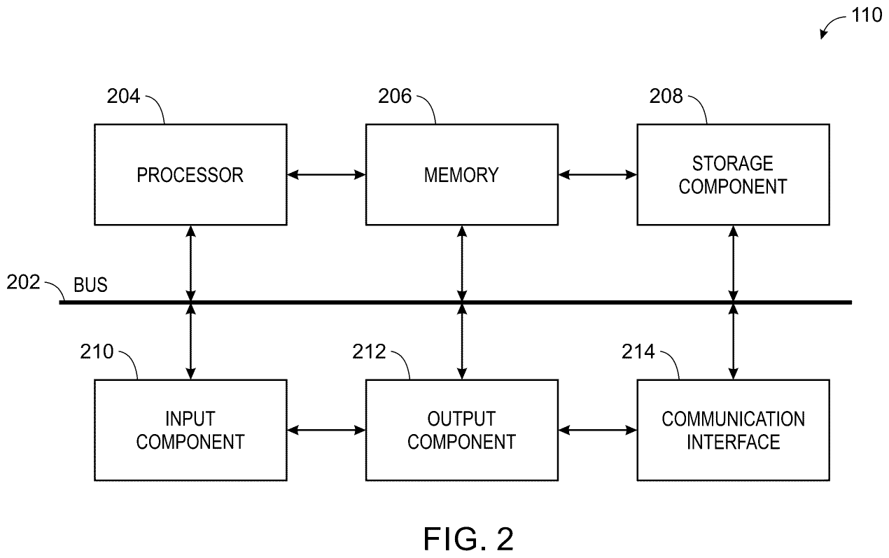

FIG. 2 is a diagram of an example deformation imaging system 110 for determining information related to cardiac deformation of an anatomical feature of a heart of a subject using deformation imaging and a plurality of overlapping segments that extend along a boundary of the anatomical feature of the heart of the subject. As shown in FIG. 2, the deformation imaging system 110 may include a bus 202, a processor 204, a memory 206, a storage component 208, an input component 210, an output component 212, and a communication interface 214.

The bus 202 includes a component that permits communication among the components of the deformation imaging system 110. The processor 204 may be implemented in hardware, firmware, or a combination of hardware and software. The processor 204 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 204 may include one or more processors capable of being programmed to perform a function. The processor 204 may include one or more processors 204 configured to perform the operations described herein. For example, a single processor 204 may be configured to perform all of the operations described herein. Alternatively, multiple processors 204, collectively, may be configured to perform all of the operations described herein, and each of the multiple processors 204 may be configured to perform a subset of the operations descried herein. For example, a first processor 204 may perform a first subset of the operations described herein, a second processor 204 may be configured to perform a second subset of the operations described herein, etc.

The memory 206 may include a random access memory (RAM), a read only memory (ROM), and/or another type of dynamic or static storage device (e.g., a flash memory, a magnetic memory, and/or an optical memory) that stores information and/or instructions for use by the processor 204.

The storage component 208 may store information and/or software related to the operation and use of the deformation imaging system 110. For example, the storage component 208 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 210 may include a component that permits the deformation imaging system 110 to receive information, such as via user input (e.g., a touch screen display, a keyboard, a keypad, a mouse, a button, a switch, a camera, and/or a microphone). Additionally, or alternatively, the input component 210 may include a sensor for sensing information (e.g., a global positioning system (GPS) component, an accelerometer, a gyroscope, and/or an actuator). The output component 212 may include a component that provides output information from the deformation imaging system 110 (e.g., a display, a speaker for outputting sound at the output sound level, and/or one or more light-emitting diodes (LEDs)).

The communication interface 214 may include a transceiver-like component (e.g., a transceiver and/or a separate receiver and transmitter) that enables the deformation imaging system 110 to communicate with other systems, such as via a wired connection, a wireless connection, or a combination of wired and wireless connections. The communication interface 214 may permit the deformation imaging system 110 to receive information from another system and/or provide information to another system. For example, the communication interface 214 may include an Ethernet interface, an optical interface, a coaxial interface, an infrared interface, a radio frequency (RF) interface, a universal serial bus (USB) interface, a Wi-Fi interface, a cellular network interface, or the like.

The deformation imaging system 110 may perform one or more processes described herein. The deformation imaging system 110 may perform these processes based on the processor 204 executing software instructions stored by a non-transitory computer-readable medium, such as the memory 206 and/or the storage component 208. A computer-readable medium may be defined herein as a non-transitory memory device. A memory device may include memory space within a single physical storage device or memory space spread across multiple physical storage devices.

The software instructions may be read into the memory 206 and/or the storage component 208 from another computer-readable medium or from another system via the communication interface 214. When executed, the software instructions stored in the memory 206 and/or the storage component 208 may cause the processor 204 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the deformation imaging system 110 may include additional components, fewer components, different components, or differently arranged components than those shown in FIG. 2. Additionally, or alternatively, a set of components (e.g., one or more components) of the deformation imaging system 110 may perform one or more functions described as being performed by another set of components of the deformation imaging system 110.

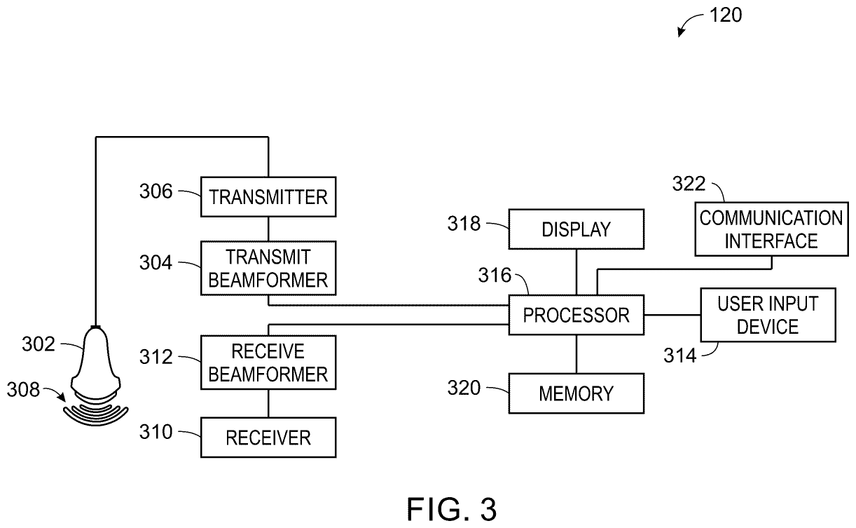

FIG. 3 is a diagram of an example ultrasound system 120 for acquiring ultrasound data of the heart of the subject. As shown in FIG. 3, the ultrasound system 120 may include an ultrasound probe 302, a transmit beamformer 304, a transmitter 306, elements 308 a receiver 310, a receive beamformer 312, a user input device 314, a processor 316, a display 318, a memory 320, and a communication interface 322. The foregoing components may be connected via wired or wireless connections.

The ultrasound probe 302 may be configured to acquire ultrasound data. For example, the ultrasound probe 302 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 302 may be configured to generate ultrasound signals, emit the ultrasound signals towards the region of interest of a subject, receive echo ultrasound signals that are back-scattered from the region of interest of the subject, generate ultrasound data based on the echo ultrasound signals, and output the ultrasound data.

The transmit beamformer 304 may be configured to apply delay times to electrical signals provided to the elements 308 to focus corresponding ultrasound signals at the region of interest. The transmitter 306 may be configured to transmit electrical signals to the elements 308 to drive the elements 308 to emit ultrasound signals towards the region of interest. The elements 308 may be configured to receive the electrical signals from the transmitter 306, convert the electrical signals into ultrasound signals, and emit the ultrasound signals towards the region of interest. The elements 308 may be configured to receive echo ultrasound signals that are back-scattered by the region of interest, convert the echo ultrasound signals into electrical signals, and provide the electrical signals to the receiver 310. The receiver 310 may be configured to receive electrical signals from the elements 308, and provide the electrical signals to the receive beamformer 312. The receive beamformer 312 may apply delay times to the electrical signals received from the elements 308.

The user input device 314 may be configured to receive a user input, and provide the user input to the processor 316. For example, the user input device 314 may be a touch screen display, a keyboard, a keypad, a mouse, a button, a switch, a microphone, or the like. Additionally, or alternatively, the user input device 314 may be configured to sense information. For example, the user input device 314 may sense information from an electro-magnetic positioning system, an inertial measurement system, an accelerometer, a gyroscope, an actuator, or the like.

The processor 316 may be configured to perform the operations as described herein. For example, the processor 216 may be a CPU, a GPU, an APU, a microprocessor, a microcontroller, a DSP, an FPGA, an ASIC, or another type of processing component. The processor 316 may be implemented in hardware, firmware, or a combination of hardware and software. The processor 316 may include one or more processors 316 configured to perform the operations described herein. For example, a single processor 316 may be configured to perform all of the operations described herein. Alternatively, multiple processors 316, collectively, may be configured to perform all of the operations described herein, and each of the multiple processors 316 may be configured to perform a subset of the operations descried herein. For example, a first processor 316 may perform a first subset of the operations described herein, a second processor 316 may be configured to perform a second subset of the operations described herein, etc.

The processor 316 may be configured to control the ultrasound probe 302 to acquire ultrasound data. The processor 316 may be configured to control which of the elements 308 are active, and control the shape of a beam emitted from the ultrasound probe 302. The processor 316 may generate ultrasound images for display. For example, the processor 216 may generate B-mode images, color Doppler images, anatomical M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 318 may be configured to display information. For example, the display 318 may be a monitor, an LED display, a cathode ray tube, a projector display, a touchscreen, tablet computer, mobile phone, or the like. The display 318 may display ultrasound images based on the ultrasound data in real-time. For example, the display 318 may display the ultrasound images within one second, two seconds, five seconds, etc., of the ultrasound data being acquired by the ultrasound probe 302.

The memory 320 may be configured to store information and/or instructions for use by the processor 316. The memory 320 may be a non-transitory computer-readable medium. For example, the memory 320 may be a random access memory (RAM), a read only memory (ROM), and/or another type of dynamic or static storage device (e.g., a flash memory, a magnetic memory, and/or an optical memory) that stores information and/or instructions for use by the processor 316. The memory 320 may be configured to store instructions that, when executed by the processor 316, cause the processor 316 to perform the operations described herein.

The communication interface 322 may be configured to enable the processor 316 to communicate with other systems, such as via a wired connection, a wireless connection, or a combination of wired and wireless connections. For example, the communication interface 322 may include an Ethernet interface, an optical interface, a coaxial interface, an infrared interface, an RF interface, a USB interface, a Wi-Fi interface, a cellular network interface, or the like.

The number and arrangement of the components of the ultrasound system 120 shown in FIG. 3 are provided as an example. In practice, the ultrasound system 120 may include additional components, fewer components, different components, or differently arranged components than those shown in FIG. 3. Additionally, or alternatively, a set of components (e.g., one or more components) of the ultrasound system 120 may perform one or more functions described as being performed by another set of components of the ultrasound system 120.

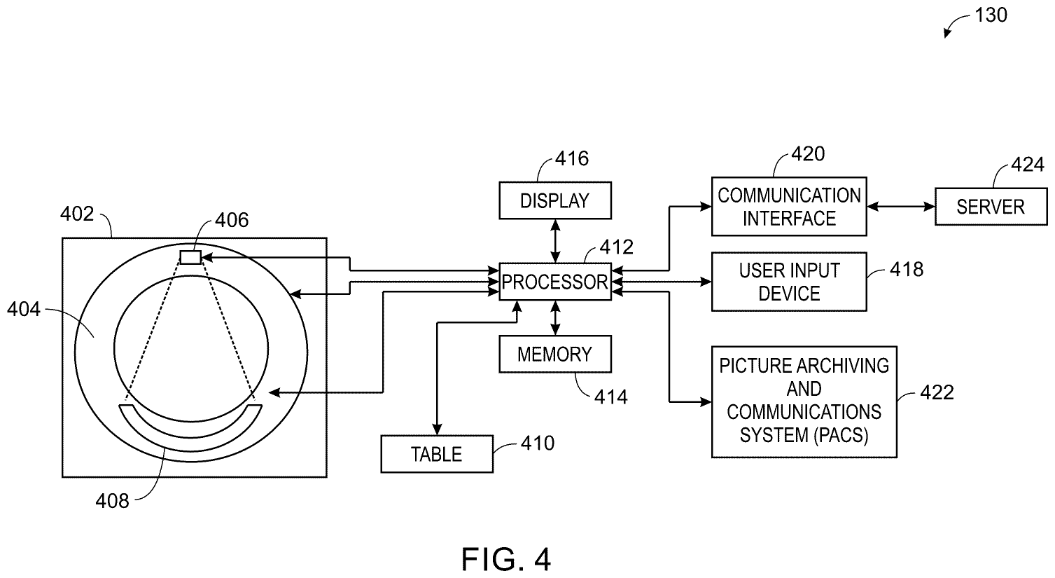

FIG. 4 is a diagram of an example preoperative imaging system 130 for acquiring preoperative imaging data of the heart of the subject. As shown in FIG. 4, the preoperative imaging system 130 may include a gantry 402, a rotational frame 404, an X-ray source 406, an X-ray detector 408, a table 410, a processor 412, a memory 414, a display 416, a user input device 418, a communication interface 420, a picture archiving and communications system (PACS) 422, and a server 424.

The processor 412 may be configured to control operations of the preoperative imaging system 130. For example, the processor 412 may be a CPU, a GPU, an APU, a microprocessor, a microcontroller, a DSP, an FPGA, an ASIC, or the like. The processor 412 may be implemented in hardware, firmware, or a combination of hardware and software. The processor 412 may include one or more processors 412 configured to perform the operations described herein. For example, a single processor 412 may be configured to perform all of the operations described herein. Alternatively, multiple processors 412, collectively, may be configured to perform all of the operations described herein, and each of the multiple processors 412 may be configured to perform a subset of the operations descried herein. For example, a first processor 412 may perform a first subset of the operations described herein, a second processor 412 may be configured to perform a second subset of the operations described herein, etc.

The processor 412 may be configured to control the gantry 402, movement of the rotational frame 404, the X-ray source 406, the X-ray detector 408, and movement of the table 410.

The memory 414 may be configured to store information and/or instructions for use by the processor 412. The memory 414 may be a non-transitory computer-readable medium. For example, the memory 414 may be a RAM, a ROM, a flash memory, a magnetic memory, an optical memory, or the like. The memory 414 may be configured to store instructions that, when executed by the processor 412, cause the processor 412 to perform the operations described herein.

The display 416 may be configured to display information. For example, the display 416 may be a monitor, an LED display, a cathode ray tube, a projector display, a touchscreen, tablet computer, mobile phone, or the like.

The user input device 418 may be configured to receive a user input, and provide the user input to the processor 412. For example, the user input device 418 may be a touch screen display, a keyboard, a keypad, a mouse, a button, a switch, a microphone, or the like. Additionally, or alternatively, the user input device 418 may be configured to sense information. For example, the user input device 418 may sense information from an electro-magnetic positioning system, an inertial measurement system, an accelerometer, a gyroscope, an actuator, or the like.

The communication interface 420 may be configured to enable the processor 412 to communicate with other devices, such as via a wired connection, a wireless connection, or a combination of wired and wireless connections. For example, the communication interface 420 may include an Ethernet interface, an optical interface, a coaxial interface, an infrared interface, an RF interface, a USB interface, a Wi-Fi interface, a cellular network interface, or the like. The PACS 422 may be configured to communicate with external systems and/or networks to permit users at various locations to access the medical image. The server 424 may be configured to store one or more models as described herein. For example, the server 424 may be an on-premises server, a cloud server, a virtual machine, or the like.

The number and arrangement of the components of the preoperative imaging system 130 shown in FIG. 4 are provided as an example. In practice, the preoperative imaging system 130 may include additional components, fewer components, different components, or differently arranged components than those shown in FIG. 4. Additionally, or alternatively, a set of components (e.g., one or more components) of the preoperative imaging system 130 may perform one or more functions described as being performed by another set of components of the preoperative imaging system 130.

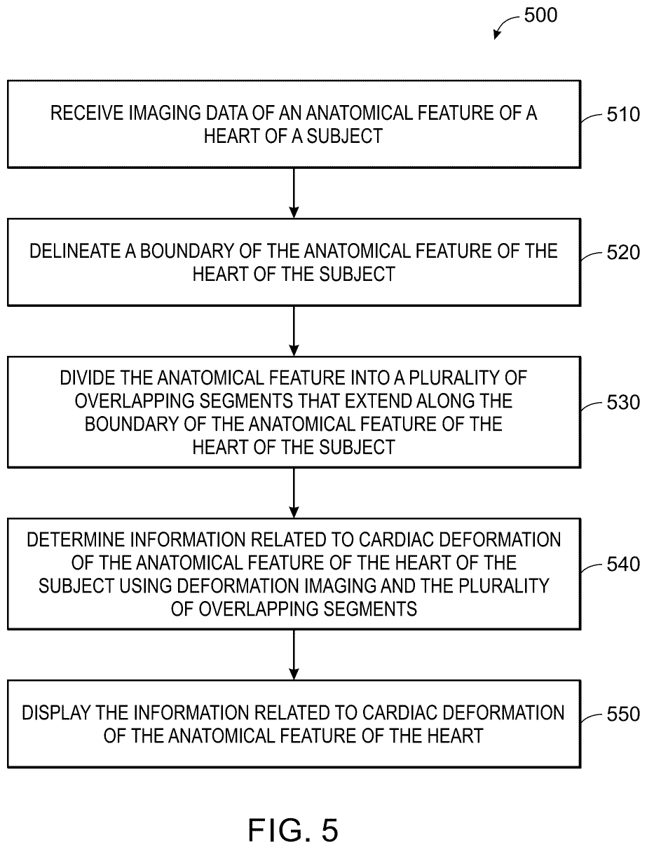

FIG. 5 is a flowchart of an example process 500 for determining information related to cardiac deformation of an anatomical feature of a heart of a subject using deformation imaging and a plurality of overlapping segments that extend along a boundary of the anatomical feature of the heart of the subject.

As shown in FIG. 5, the process 500 may include receiving imaging data of an anatomical feature of a heart of a subject (operation 510). For example, the deformation imaging system 110 may receive imaging data from the ultrasound system 120, the preoperative imaging system 130, or the like. The imaging data may be ultrasound data, CT data, MRI data, X-ray data, PET data, or the like. The anatomical feature of the heart of the subject may be the left atrium, the right atrium, the left ventricle, the right ventricle, the mitral valve, the aortic valve, or the like. The imaging data may be a 2D medical image, a 3D medical image, or the like.

As further shown in FIG. 5, the process 500 may include delineating a boundary of the anatomical feature of the heart of the subject (operation 520). For example, the deformation imaging system 110 may delineate a boundary of the anatomical feature of the heart of the subject using a segmentation model, an image processing technique, an artificial intelligence (AI) model, or the like. The deformation imaging system 110 may be configured to delineate the boundary of the anatomical feature based on detecting one or more portions of the anatomical feature of the heart. The one or more portions may be the myocardium, the epicardial border, the endocardial border, the myocardial mid-line, or the like.

As further shown in FIG. 5, the process 500 may include dividing the anatomical feature into a plurality of overlapping segments that extend along the boundary of the anatomical feature of the heart of the subject (operation 530). For example, the deformation imaging system 110 may divide the anatomical feature into a plurality of overlapping segments that extend along the boundary of the anatomical feature of the heart of the subject automatically, via a user input, or the like.

A segment of the anatomical feature of the heart may be any portion of the anatomical feature of the heart. For instance, a segment may represent myocardium within a particular boundary. For example, the left atrium may include a left-basal segment, a left-middle segment, a left-apical segment, a right-apical segment, a right-middle segment, a right-basal segment, or the like. A segment may include any portion, or combination of portions, of the foregoing segments. The contours of the segment may be delineated by the boundary of the anatomical feature. The segment may be a 2D segment or a 3D segment.

According to an embodiment, the deformation imaging system 110 may be configured to automatically divide the anatomical feature into the plurality of overlapping segments. The deformation imaging system 110 may be configured with segment information that identifies a length of each segment, that identifies a number of segments, that identifies an amount of overlap of each segment, or the like. The deformation imaging system 110 may divide the anatomical feature into the plurality of overlapping segments that extend along the boundary of the anatomical feature of the heart of the subject based on the segment information.

According to an embodiment, the deformation imaging system 110 may divide the anatomical feature into the plurality of overlapping segments via a sliding mechanism configured to traverse the entirety of the anatomical feature. For example, the deformation imaging system 110 may provide a segment window at an initial position, and incrementally shift the segment window across the anatomical feature along the boundary of the anatomical feature. In this case, the initial position may be a basal point of the anatomical feature. The segment window may define the positions of the overlapping segments at each incremental shift of the segment window. For example, the first (or initial) position of the segment window may identify a position of a first segment, the second position of the segment window may identify a position of the second segment, the third position of the segment window may identify a position of the third segment, etc. The shift of the segment window may define an amount of overlap of adjacent segments. For example, if a first segment includes a length of 4 centimeters (cm) and the segment window is shifted 2 cm to define the second segment, then the first segment and the second segment include 2 cm of overlap.

According to an embodiment, the deformation imaging system 110 may be configured to divide the anatomical feature into the plurality of overlapping segments based on a user input received via a user interface. For example, the deformation imaging system 110 may receive, via the user interface, one or more user inputs that delineate the plurality of overlapping segments of the anatomical feature of the heart.

According to an embodiment, the one or more user inputs that delineate the plurality of overlapping segments of the anatomical feature of the heart may delineate a number of the plurality of overlapping segments, respective sizes of the plurality of overlapping segments, respective positions of the plurality of overlapping segments, amounts of overlap between the plurality of overlapping segments, or the like. For example, the user may interact with the user interface to input one or more user inputs that delineate the plurality of overlapping segments in relation to the anatomical feature of the heart. In this case, the one or more user inputs may delineate the number of the plurality of overlapping segments, the sizes of the plurality of overlapping segments, the positions of the plurality of overlapping segments, the amounts of overlap between the plurality of overlapping segments, or the like.

Additionally, or alternatively, the user may interact with the user interface to input the one or more user inputs that manipulate a segment window on the user interface. For example, the one or more user inputs may position the segment window along the anatomical feature, may slide the segment window along the anatomical feature, may elongate, or shorten, the segment window, or the like. In this case, the number of user interactions may delineate the number of the plurality of overlapping segments, the sizes of the plurality of overlapping segments, the positions of the plurality of overlapping segments, the amounts of overlap between the plurality of overlapping segments, or the like. Additionally, or alternatively, the user may interact with the user interface to input one or more user inputs that delineate a discrete number of the plurality of overlapping segments, discrete size of the plurality of overlapping segments, discrete positions of the plurality of overlapping segments, discrete amounts of overlap between the plurality of overlapping segments, or the like.

As further shown in FIG. 5, the process 500 may include determining information related to cardiac deformation of the anatomical feature of the heart of the subject using deformation imaging and the plurality of overlapping segments (operation 540). For example, the deformation imaging system 110 may determine information related to cardiac deformation of the anatomical feature of the heart of the subject using deformation imaging.

The deformation imaging system 110 may be configured to perform deformation imaging using a deformation imaging technique, such as a template matching technique (e.g., speckle tracking), an image registration technique, an image segmentation technique, an AI technique, or the like. The deformation imaging system 110 may be configured to perform deformation imaging using imaging data from the ultrasound system 120, the preoperative imaging system 130, or the like. The imaging data may be ultrasound data, CT data, MRI data, X-ray data, PET data, or the like.

The information related to cardiac deformation of the anatomical feature of the heart of the subject may be strain values of the plurality of overlapping segments, amounts of time to reach maximum strain values of the respective segments, particular segments that are associated with amounts of time that are greater than or less than respective thresholds, a mechanical dispersion value, a cardiac mechanical dyssynchrony parameter, or the like.

The deformation imaging system 110 may identify the respective segments of the anatomical feature of the heart based on dividing the anatomical feature into the plurality of overlapping segments. Further, the deformation imaging system 110 may track the respective segments over time using deformation imaging based on identifying the plurality of overlapping segments. Further still, the deformation imaging system 110 may determine respective strain values of the plurality of overlapping segments over time based on tracking the respective segments using deformation imaging. For example, the deformation imaging system 110 may determine a strain value based on a starting length of a segment and a final length of the segment. As an example, if the starting length of the segment is “10” and the final length of the segment is “8,” then the deformation imaging system 110 may determine a strain value of “−20%.” As another example, if the starting length of the segment is “8” and the final length of the segment is “10,” then the deformation imaging system 110 may determine a strain value of “20%. ” Further still, the deformation imaging system 110 may determine respective amounts of time for each of the segments to respectively reach maximum strain values. The deformation imaging system 110 may determine a mechanical dispersion value based on the respective amounts of time for each of the segments to respectively reach maximum strain values. For example, the deformation imaging system 110 may determine the mechanical dispersion value based on a standard deviation of the amounts of time.

As further shown in FIG. 5, the process 500 may include displaying the information related to cardiac deformation of the anatomical feature of the heart (operation 550). For example, the deformation imaging system 110 may display the information related to cardiac deformation of the anatomical feature of the heart, such as the mechanical dispersion value, strain values of the set of segments, amounts of time to reach maximum strain values of the respective segments, particular segments that are associated with amounts of time that are greater than or less than respective thresholds, or the like.

Although FIG. 5 describes the delineation of a single boundary of a single anatomical feature and the determination of information related to cardiac deformation of the single anatomical feature, it should be understood that the deformation imaging system 110 may delineate respective boundaries of multiple anatomical features of the heart and determination information related to cardiac deformation of the multiple anatomical features of the heart. For example, the deformation imaging system 110 may delineate the boundary of the left atrium, the boundary of the left ventricle, the boundary of the right atrium, the boundary of the right ventricle, and/or the like, and determine information related to cardiac deformation of the multiple anatomical features of the heart.

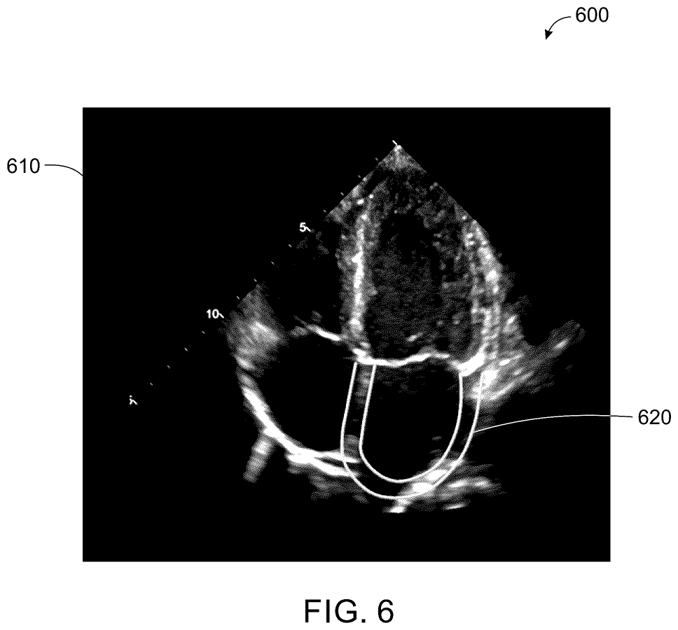

FIG. 6 is a diagram of an example user interface 600 that displays an ultrasound image and an anatomical feature. As shown, the user interface 600 may display an ultrasound image 610 of the heart, and may display an anatomical feature 620 of the heart with a delineated boundary. The anatomical feature 620 of the heart may represent a region of interest for determining information related to cardiac deformation. It should be understood that the deformation imaging system 110 may delineate multiple boundaries of multiple anatomical features. Further, although an ultrasound image is shown, it should be understood that the deformation imaging system 110 may use other types of medical images associated with different imaging modalities.

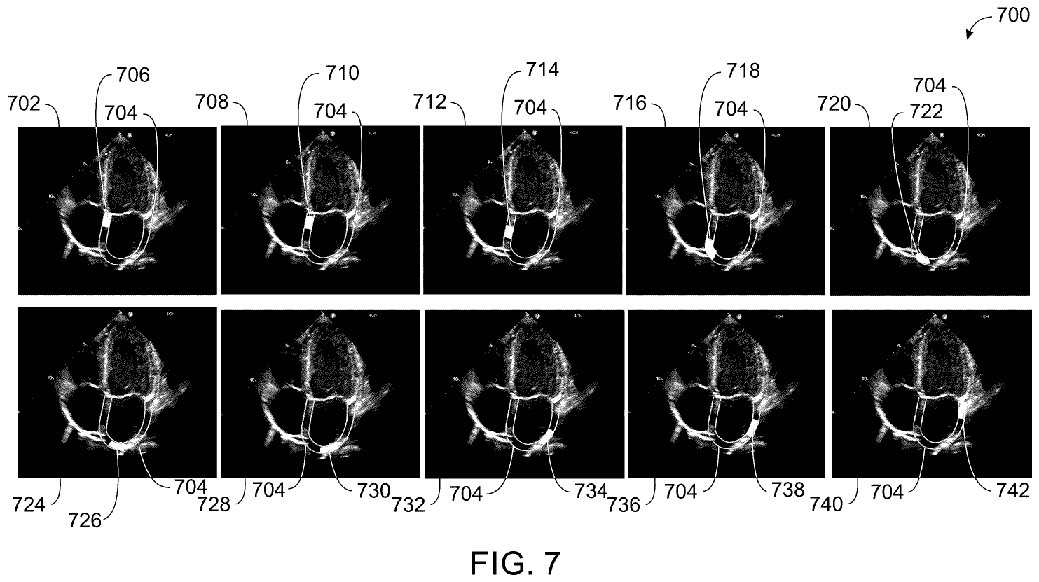

FIG. 7 is a diagram of an example user interface 700 that displays an ultrasound image, an anatomical feature with a delineated boundary, and a segment window that is incrementally shifted across the anatomical feature along the boundary of the anatomical feature. As shown, the user interface 700 may display an ultrasound image 702 that includes an anatomical feature 704. The user interface 700 may also display a segment 706 in relation to the anatomical feature 704. The deformation imaging system 110 may shift the segment window along the boundary of the anatomical feature 704. In this case, the user interface 700 may iteratively display the ultrasound image 708 that displays the segment 710, the ultrasound image 712 that displays the segment 714, the ultrasound image 716 that displays the segment 718, the ultrasound image 720 that displays the segment 722, the ultrasound image 724 that displays the segment 726, the ultrasound image 728 that displays the segment 730, the ultrasound image 732 that displays the segment 734, the ultrasound image 736 that displays the segment 738, and the ultrasound image 740 that displays the segment 742. The deformation imaging system 110 may divide the anatomical feature into the plurality of overlapping segments 706, 710, 714, 718, 722, 726, 730, 734, 738, and 742 based on one or more user inputs. Alternatively, the deformation imaging system 110 may automatically divide the anatomical feature into the plurality of overlapping segments 706, 710, 714, 718, 722, 726, 730, 734, 738, and 742 by incrementally shifting the segment window.

As shown in FIG. 7, the segment 710 may overlap the segment 706, the segment 714 may overlap the segment 710, the segment 718 may overlap the segment 714, the segment 722 may overlap the segment 718, the segment 726 may overlap the segment 722, the segment 730 may overlap the segment 726, the segment 734 may overlap the segment 730, the segment 738 may overlap the segment 734, and the segment 742 may overlap the segment 738. According to another embodiment, the deformation imaging system 110 may display a 3D medical image, and 3D segments. For example, the 3D segments may be overlapping segments that extend on the anatomical feature, and that can be manipulated around and on the anatomical feature.

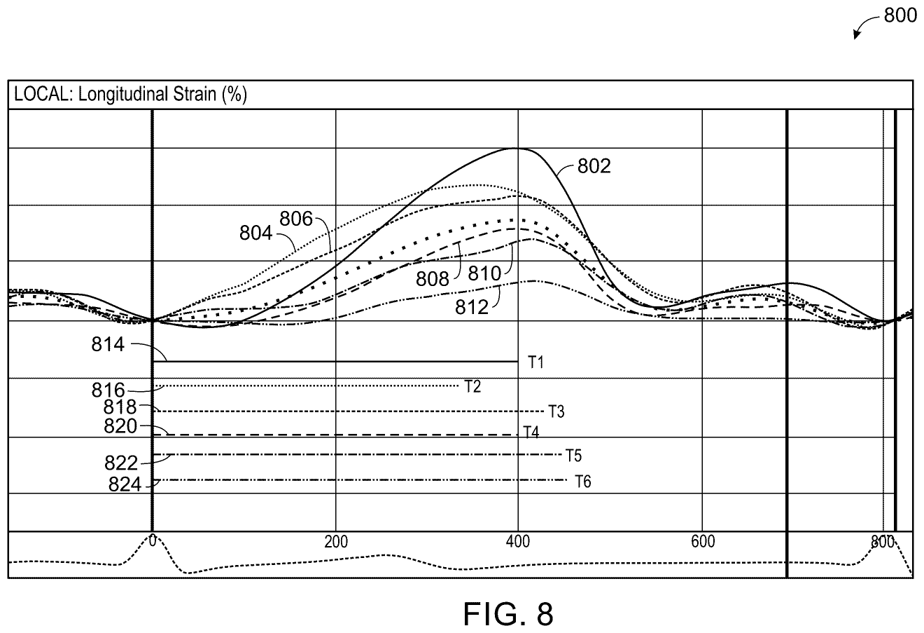

FIG. 8 is a diagram of an example user interface 800 that displays strain traces that display respective strain values of a set of corresponding segments over time, and time plots that display respective amounts of time to reach maximum strain values for the set of corresponding segments. As shown, the user interface 800 may display a first strain trace 802 that displays strain values of a first segment over time, display a second strain trace 804 that displays strain values of a second segment over time, display a third strain trace 806 that displays strain values of a third segment over time, display a fourth strain trace 808 that displays strain values of a fourth segment over time, display a fifth strain trace 810 that displays strain values of a fifth segment over time, and display a sixth strain trace 812 that displays strain values of a sixth segment over time. Further, the user interface 800 may display a first time plot 814 that displays an amount of time for the first segment to reach a maximum strain value, a second time plot 816 that displays an amount of time for the second segment to reach a maximum strain value, a third time plot 818 that displays an amount of time for the third segment to reach a maximum strain value, display a fourth time plot 820 that displays an amount of time for the fourth segment to reach a maximum strain value, display a fifth time plot 822 that displays an amount of time for the fifth segment to reach a maximum strain value, and display a sixth time plot 824 that displays an amount of time for the sixth segment to reach a maximum strain value.

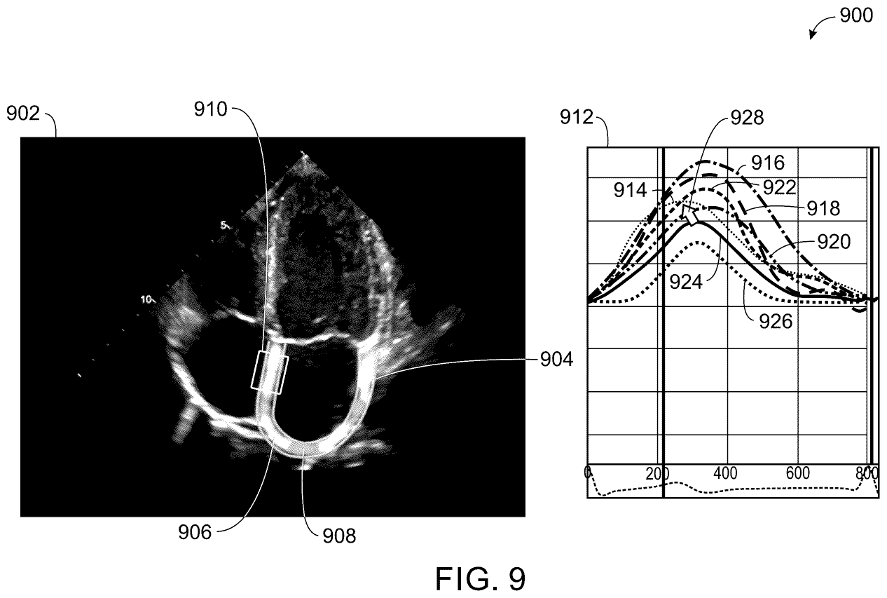

FIG. 9 is a diagram of an example user interface 900 that displays an ultrasound image and respective tracking quality indicators of a set of segments, and that displays strain traces that display respective strain values of a set of corresponding segments over time. As shown, the user interface 900 may display an ultrasound image 902, and an anatomical feature 904 with a delineated boundary, a region 906 that is constituted by one or more segments having tracking qualities that are greater than a tracking quality threshold, and a region 908 that is constituted by one or more segments having tracking qualities that are less than the tracking quality threshold. The user interface 900 may visually differentiate the regions by using different image parameters (e.g., colors, hues, opacities, patterns, or the like). Further, as shown, the user interface 900 may display a strain trace plot 912 that displays a first strain trace 914 of a first segment, a second strain trace 916 of a second segment, a third strain trace of a third segment 918, a fourth strain trace 920 of a fourth segment, a fifth strain trace 922 of a fifth segment, a sixth strain trace 924 of a sixth segment, and a seventh strain trace of a seventh segment 926. Further, as shown in FIG. 9, the user interface 900 may display an icon 910 on the ultrasound image 902 that permits a user to select a particular segment, segments, or a portion of a segment. Based on the position of the icon 910, the user interface 900 may display an identifier 928 on the strain trace plot 912 that highlights a particular segment that corresponds to the icon 910. The deformation imaging system 110 may determine a tracking quality of a segment for speckle tracking echocardiography, and display information identifying the tracking quality of the segment. The deformation imaging system 110 may permit the user to select a particular strain trace, and remove the strain trace from the determination of the information related to cardiac deformation. In this way, a user can identify a segment associated with low tracking quality, and exclude a strain trace associated with the segment from being used in the determination of the information related to cardiac deformation. Alternatively, user can override the system decision and approve a rejected region (segment) or vice versa.

According to another embodiment, the deformation imaging system 110 may display a bounding box illustrating a sub-segment that was used to determine information related to cardiac deformation of a region including a segment. In this way, the deformation imaging system 110 may allow for a more detailed understanding of how strain values are derived from particular areas within a larger segmented region.

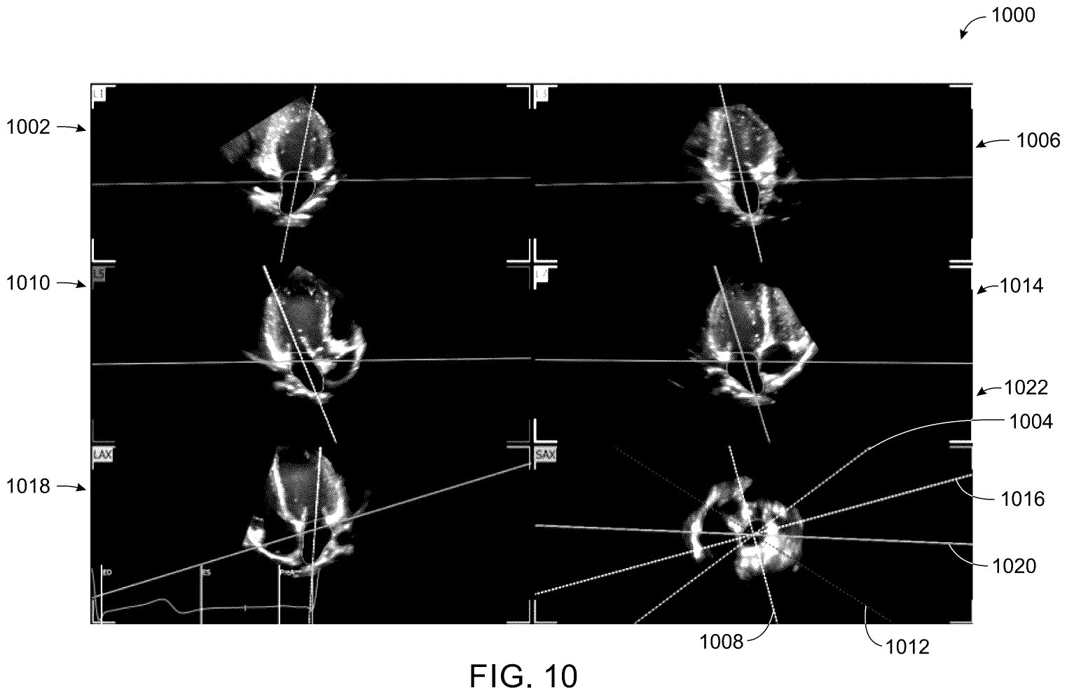

FIG. 10 is a diagram of an example user interface that displays ultrasound images corresponding to respective apical planes of the heart. As shown, the user interface 1000 may display a first ultrasound image 1002 corresponding to a first apical plane 1004, a second ultrasound image 1006 corresponding to a second apical plane 1008, a third ultrasound image 1010 corresponding to a third apical plane 1012, a fourth ultrasound image 1014 corresponding to a fourth apical plane 1016, a fifth ultrasound image 1018 corresponding to a fifth apical plane 1020, and a sixth ultrasound image 1022 that displays the first apical plane 1004, the second apical plane 1008, the third apical plane 1012, the fourth apical plane 1016, and the fifth apical plane 1020. The deformation imaging system 110 may acquire ultrasound data for the multiple apical planes, and determine the information related to cardiac deformation of the anatomical feature of the heart for the different apical planes using the corresponding ultrasound data.

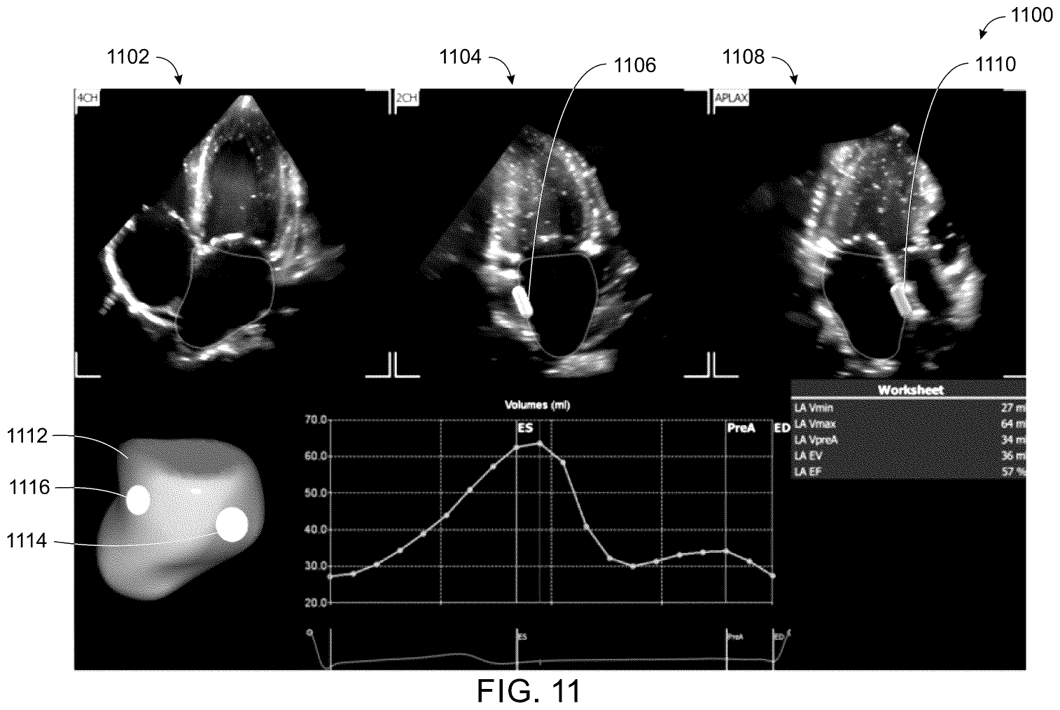

FIG. 11 is a diagram of an example user interface that displays ultrasound images displaying respective markers of segments associated with amounts of time to reach maximum strain value that are greater than or less than respective thresholds, and that displays a model of the anatomical feature of the heart that displays the markers. As shown, the user interface 1100 may display an ultrasound image 1102 of the heart, an ultrasound image 1104 that displays a first marker 1106 that identifies a segment of the anatomical feature of the heart that is associated with a greatest amount of time to reach a maximum strain value, and an ultrasound image 1108 that displays a second marker 1110 that identifies a segment of the anatomical feature of the heart that is associated with a least amount of time to reach a maximum strain value. Further, as shown, the user interface 1100 may display a model 1112 of the anatomical feature of the heart that displays a first icon 1114 corresponding to the first marker 1106, and that displays a second icon 1116 corresponding to the second marker 1110. The deformation imaging system 110 may determine a segment that includes an amount of time to reach a maximum strain value that is greater than a threshold, and display information identifying the segment. Additionally, or alternatively, the deformation imaging system 110 may determine a segment that includes an amount of time to reach a maximum strain value that is less than a threshold, and display information identifying the segment. For example, the deformation imaging system 110 may display the segments in a particular color, may highlight the segments, may display the segments in a bounding box, or the like.

Alternatively, a user can interact with the user interface 1100 to place a marker in relation to the model 1112. The deformation imaging system 110 may highlight a corresponding segment in an ultrasound image based on the user input. Further, the deformation imaging system 110 may display an indication of a time for the segment to reach a maximum strain value. For example, the deformation imaging system 110 may display an indication of the time, may adjust an image parameter of the segment (e.g., color, brightness, hue, etc.), or the like. Additionally, or alternatively, a user can interact with the user interface 1100 to move a marker in relation to the model 1112. Based on the user input, the deformation imaging system 110 may highlight a corresponding segment in the ultrasound image.

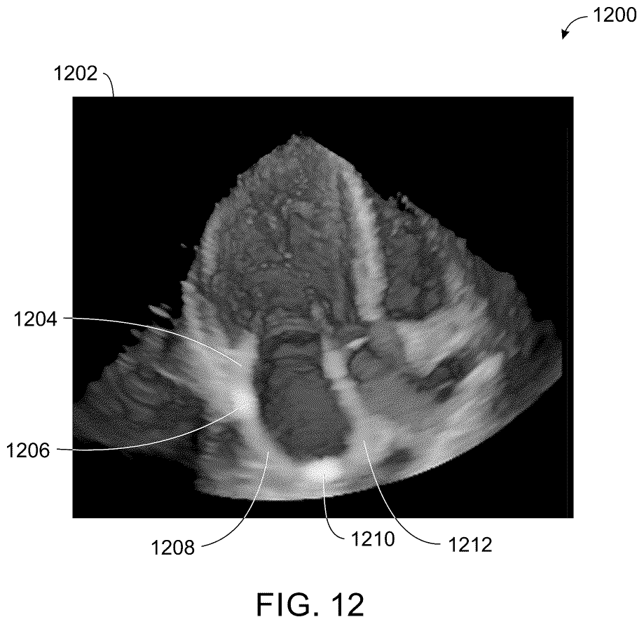

FIG. 12 is a diagram of an example user interface that displays an ultrasound image displaying respective markers of segments that identify respective times of the corresponding segments to reach peak strain values. For instance, the deformation imaging system 110 may determine respective times for each of the segments to reach peak (or maximum) strain values, and may display markers that identify the respective times for each of the segments to reach peak strain values. As shown, the user interface 1200 may display an ultrasound image 1202 that depicts an anatomical feature of the heart. The user interface 1200 may display a first region 1204 that includes one or more segments that are associated with amounts of time to reach peak (or maximum) strain values that are relatively lower than other segments. Further, user interface 1200 may display a second region 1206 that includes one or more segments that are associated with amounts of time to reach maximum strain values that are relatively greater than other segments. Further, the user interface 1200 may display a third region 1208 that includes one or more segments that are associated with amounts of time to reach maximum strain values that are relatively lower than other segments. Further, the user interface 1200 may display a fourth region 1210 that includes one or more segments that are associated with amounts of time to reach maximum strain values that are relatively greater than other segments. Further, the user interface 1200 may display a fifth region 1212 that includes one or more segments that are associated with amounts of time to reach maximum strain values that are relatively lower than other segments. Alternatively, the user interface 1200 may adjust an image parameter for the entire ultrasound image 1202 based on the time for the one or more segments to reach maximum strain values. The user interface 1200 may use various colorization schemes with different color maps. Additionally, or alternatively, the deformation imaging system 110 may determine amounts of time for all segments to reach their peak (or maximum) strain values, and place markers next to those segments that take a longer time to reach the peak value.

As an alternative, the user interface 1200 may display a first region 1204 that includes one or more segments that are associated with amounts of time to reach maximum strain values that are less than a time threshold. Further, user interface 1200 may display a second region 1206 that includes one or more segments that are associated with amounts of time to reach maximum strain values that are greater than the time threshold. Further, the user interface 1200 may display a third region 1208 that includes one or more segments that are associated with amounts of time to reach maximum strain values that are less than the time threshold. Further, the user interface 1200 may display a fourth region 1210 that includes one or more segments that are associated with amounts of time to reach maximum strain values that are greater than the time threshold. Further, the user interface 1200 may display a fifth region 1212 that includes one or more segments that are associated with amounts of time to reach maximum strain values that are less than the time threshold. Alternatively, the user interface 1200 may adjust an image parameter for the entire ultrasound image 1202 based on the time for the one or more segments to reach maximum strain values. The user interface 1200 may use various colorization schemes with different color maps.

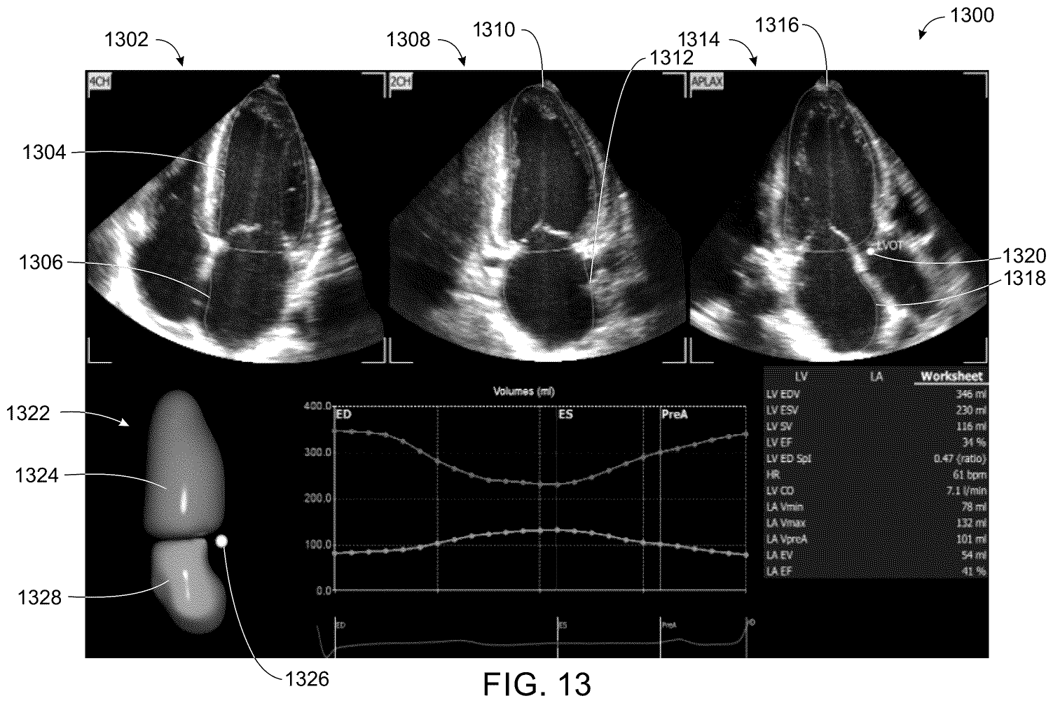

FIG. 13 is a diagram of an example user interface that displays ultrasound images displaying multiple anatomical features of the heart, and that displays a model displaying the multiple anatomical features of the heart. As shown, the user interface 1300 may display a first ultrasound image 1302 that displays a first anatomical feature 1304 of the heart and a second anatomical feature 1306 of the heart, display a second ultrasound image 1308 that displays a first anatomical feature 1310 of the heart and a second anatomical feature 1312 of the heart, display a third ultrasound image 1314 that displays a first anatomical feature 1316 of the heart, a second anatomical feature 1318 of the heart, and a marker 1320 that identifies a portion of the second anatomical feature 1318 of the heart that is associated with an amount of time to reach a maximum strain value that is greater than a threshold. Further, as shown, the user interface 1300 may display a model 1322 that displays the first anatomical feature 1324, the second anatomical feature 1326, and a marker 1328 that identifies a portion of the second anatomical feature 1326 of the heart that is associated with an amount of time to reach a maximum strain value that is greater than a threshold. The deformation imaging system 110 may determine information related to cardiac deformation of multiple anatomical features of the heart, and display the information related to the cardiac deformation of the multiple anatomical features of the heart.

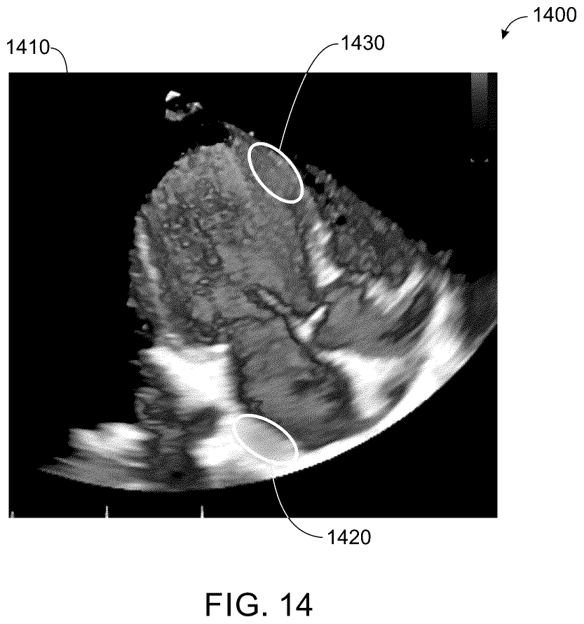

FIG. 14 is a diagram of an example user interface 1400 that displays an ultrasound image with markers that highlights areas of longest contraction or conduction delay of the heart. As shown, the user interface 1100 may display an ultrasound image 1410 that displays a marker 1420 and a marker 1430 that highlight areas of longest contraction or conduction delay of the heart. The deformation imaging system 110 may determine the areas of longest contraction or conduction delay of the heart based on the information related to the cardiac deformation of the of the heart, and display the markers that highlight the areas of longest contraction or conduction delay of the heart. The deformation imaging system 110 may determine the areas based on measurements of one or more anatomical features of the heart (e.g., the left ventricle, the left atrium, the right ventricle, the right atrium, etc.).

According to an embodiment, the deformation imaging system 110 may display 3D ultrasound data that is color-coded based on the amount of time it takes for a respective segment to reach its peak contraction state. Therefore, in addition to 3D B-mode data, the deformation imaging system 110 may visualize 3D color-coded ultrasound data based on contraction time.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A system comprising:
a memory configured to store instructions; and
one or more processors configured to execute the instructions to:
receive imaging data of an anatomical feature of a heart of a subject;
delineate a boundary of the anatomical feature of the heart of the subject;
divide the anatomical feature into a plurality of overlapping segments that extend along the boundary of the anatomical feature of the heart of the subject;
determine information related to cardiac deformation of the anatomical feature of the heart of the subject using deformation imaging and the plurality of overlapping segments; and
display the information related to cardiac deformation of the anatomical feature of the heart.

2. The system of claim 1, wherein the information related to cardiac deformation of the anatomical feature of the heart includes a mechanical dispersion value.

3. The system of claim 1, wherein the information related to cardiac deformation of the anatomical feature of the heart includes strain values of the plurality of overlapping segments.

4. The system of claim 1, wherein the one or more processors are further configured to:
receive one or more user inputs that delineate a number of the plurality of overlapping segments, respective positions of the plurality of overlapping segments in relation to the anatomical feature of the heart, or respective amounts of overlap between the plurality of overlapping segments.

5. The system of claim 1, wherein delineating the boundary of the anatomical feature of the heart of the subject comprises delineating the boundary of the anatomical feature of the heart of the subject by segmenting the anatomical feature.

6. The system of claim 1, wherein the information related to cardiac deformation of the anatomical feature of the heart of the subject includes respective amounts of time of the plurality of overlapping segments to reach peak contraction states, and wherein the one or more processors are further configured to:
display 3D ultrasound data that is color-coded based on the amounts of time of the plurality of overlapping segments to reach peak contraction states.

7. The system of claim 1, wherein the one or more processors are further configured to:
determine information related to cardiac deformation of multiple anatomical features of the heart.

8. A method comprising:
receiving imaging data of an anatomical feature of a heart of a subject;
delineating a boundary of the anatomical feature of the heart of the subject;
dividing the anatomical feature into a plurality of overlapping segments that extend along the boundary of the anatomical feature of the heart of the subject;
determining information related to cardiac deformation of the anatomical feature of the heart of the subject using deformation imaging and the plurality of overlapping segments; and
displaying the information related to cardiac deformation of the anatomical feature of the heart.

9. The method of claim 8, wherein the information related to cardiac deformation of the anatomical feature of the heart includes a mechanical dispersion value.

10. The method of claim 8, wherein the information related to cardiac deformation of the anatomical feature of the heart includes strain values of the plurality of overlapping segments.

11. The method of claim 8, further comprising:
receiving one or more user inputs that delineate a number of the plurality of overlapping segments, respective positions of the plurality of overlapping segments in relation to the anatomical feature of the heart, or respective amounts of overlap between the plurality of overlapping segments.

12. The method of claim 8, wherein delineating the boundary of the anatomical feature of the heart of the subject comprises delineating the boundary of the anatomical feature of the heart of the subject by segmenting the anatomical feature.

13. The method of claim 8, further comprising:
displaying strain traces of the plurality of overlapping segments.

14. The method of claim 8, further comprising:
determining information related to cardiac deformation of multiple anatomical features of the heart.

15. A non-transitory computer-readable medium storing instructions that, when executed by one or more processors, cause the one or more processors to:
receive imaging data of an anatomical feature of a heart of a subject;
delineate a boundary of the anatomical feature of the heart of the subject;
divide the anatomical feature into a plurality of overlapping segments that extend along the boundary of the anatomical feature of the heart of the subject;
determine information related to cardiac deformation of the anatomical feature of the heart of the subject using deformation imaging and the plurality of overlapping segments; and
display the information related to cardiac deformation of the anatomical feature of the heart.

16. The non-transitory computer-readable medium of claim 15, wherein the information related to cardiac deformation of the anatomical feature of the heart includes a mechanical dispersion value.

17. The non-transitory computer-readable medium of claim 15, wherein the information related to cardiac deformation of the anatomical feature of the heart includes strain values of the plurality of overlapping segments.

18. The non-transitory computer-readable medium of claim 15, wherein the instructions further cause the one or more processors to:
receive one or more user inputs that delineate a number of the plurality of overlapping segments, respective positions of the plurality of overlapping segments in relation to the anatomical feature of the heart, or respective amounts of overlap between the plurality of overlapping segments.

19. The non-transitory computer-readable medium of claim 15, wherein delineating the boundary of the anatomical feature of the heart of the subject comprises delineating the boundary of the anatomical feature of the heart of the subject by segmenting the anatomical feature.

20. The non-transitory computer-readable medium of claim 15, wherein the instructions further cause the one or more processors to:
displaying strain traces of the plurality of overlapping segments.
